(19) United States
(12) Patent Application Publication
(10) Pub. No.: US 2024/0009160 A1
(43) Pub. Date: Jan. 11, 2024

(54) USE OF (S)-3-AMINO-4-(DIFLUOROMETHYLENYL)CYCLOPENT-1-ENE-1-CARBOXYLIC ACID IN THE TREATMENT OF CANCER

(71) Applicant: Ovid Therapeutics, Inc., New York, NY (US)

(72) Inventor: Aaron Goldman, New York, NY (US)

(21) Appl. No.: 18/347,732

(22) Filed: Jul. 6, 2023

Related U.S. Application Data
(60) Provisional application No. 63/358,677, filed on Jul. 6, 2022.

(51) Int. Cl. A61K 31/197; A61K 9/00

(52) U.S. Cl. CPC A61K 31/197; A61K 9/0053

(57) ABSTRACT

Methods and compositions including (S)-3-amino-4-(difluoromethylenyl)cyclopent-1-ene-1-carboxylic acid or a pharmaceutically acceptable salt thereof are provided for use in treating cancer and inhibiting cancer proliferation and metastasis.

### CROSS REFERENCE TO RELATED APPLICATIONS

This application claims benefit of and priority to U.S. Provisional Application No. 63/358,677, filed on Jul. 6, 2022, and which is incorporated herein by reference in its entirety.

### TECHNICAL FIELD

Treatment of cancer with (S)-3-amino-4-(difluoromethylenyl) cyclopent-1-ene-1-carboxylic acid or pharmaceutically acceptable salts thereof.

### BACKGROUND

According to the World Health Organization, (https://www.who.int/news-room/fact-sheets/detail/cancer) cancer is a generic term for a large group of diseases that can affect any part of the body. Other terms used are malignant tumors and neoplasms. Cancer is a leading cause of death worldwide, accounting for nearly 10 million deaths in 2020, or nearly one in six deaths. A defining feature of cancer is the rapid creation of abnormal cells that grow beyond their usual boundaries, and which can then invade adjoining parts of the body and spread to other organs; the latter process is referred to as metastasis. Widespread metastases are the primary cause of death from cancer.

Medulloblastoma (MB) is the most common malignant pediatric brain tumor. According to OMIM #155255, MB accounts for 16% of all pediatric brain tumors, and 40% of all cerebellar tumors in childhood are MB. Approximately 10 to 15% of medulloblastomas are diagnosed in infancy. MB occurs bimodally, with peak incidences between 3 and 4 years and 8 and 9 years of age. MB accounts for less than 1% of central nervous system (CNS) tumors in adults, with highest incidence in adults 20 to 34 years of age.

MB is defined by four molecular subgroups (Wnt, Shh, Group 3, Group 4) based on transcriptional and epigenetic profiles. See, Manoranjan, et al. Nat Commun 11, 4323 (2020). The Wnt (wingless) and Shh (Sonic Hedgehog) MBs are named for the signaling pathways thought to play prominent roles in the pathogenesis of that subgroup. The Wnt signaling pathways are a group of signal transduction pathways which begin with proteins that pass signals into a cell through cell surface receptors. NT-activated medulloblastomas are defined by activating mutations in the WNT/I3-catenin signaling pathway (e.g., CTNNB1 or germline APC), are often associated with loss of chromosome 6, and have the most favorable prognosis. Manoranjan, et al., supra. Wnt MB accounts for 10% of cases. Id.

The Shh signaling pathway is one of the major trafficking networks that regulates the key events during developmental processes, i.e., growth and patterning of multicellular embryos. Aberration in the regulation and transduction of the Shh signaling pathway has been implicated in birth defects, tissue regeneration, stem cell renewal and cancer growth. Choudhry et al., Ann Neurosci. 2014; 21(1):28-31. SHH-activated medulloblastomas (SHH-MB) account for 25-30% of all MBs and occur with a bimodal age distribution, encompassing many infant and adult, but fewer childhood cases. Menyhart and Gyorffy, Annals of Clinical and Translational Neurology 2019; 6(5): 990-1005. Different age groups are characterized by distinct survival outcomes and age-specific alterations of regulatory pathways. Id.

Group 3 MB has a photoreceptor/retinal expression signature, while group 4 MB expresses neuronal genes. Tamayo-Orrego, Lukas, and Frédéric Charron. 1000Research vol. 8 F1000 Faculty Rev-1823. 29 Oct. 2019. The current idea is that each different MB group is derived from a specific cell of origin, which therefore determines the clinical and molecular behavior of the disease. Id.

Glioma is a type of brain tumor that develops from the glial cells, which are specialized cells that surround and support neurons in the brain. According to the National Institutes of Health (NIH), Genetic and Rare Diseases Information Center (GARD), glioma is generally classified by the type of glia cell that is involved in the tumor: astrocytoma-tumors that develop from star shaped glial cells called astrocytes; ependymomas-tumors that arise from ependymal cells that line the ventricles of the brain and the center of the spinal cord; oligodendrogliomas-tumors that affect the oligodendrocytes, the myelinating glia of the central nervous system. Gliomas are graded on a scale of one to four. Grade one gliomas usually grow slowly and frequently behave in a more benign fashion. Grade two and grade three gliomas can grow more quickly and they frequently require more aggressive treatment. Grade four gliomas are the most aggressive type and are also known as glioblastoma. Symptoms of glioma vary by type but may include headaches, nausea and vomiting, confusion, personality changes, trouble with balance, vision problems, speech difficulties, and/or seizures. In studies, 8 members of 19 WNTs, i.e., WNT1, 2, 2B, 3A, 5A, 6, 7A and 7B, were shown as being related with glioma development. Xu et al., Bioscience reports vol. 40 (3) (2020).

Breast cancer originates in breast tissue and cells usually form a tumor which may be in situ or invasive. According to the NIH, breast cancer is the second most common type of cancer in women in the United States. Rarely, it can also affect men. Certain genetic markers are associated with the risk of breast cancer such as BRCA1, BRCA2 and HER2 which may raise the risk of ovarian and other cancers. The different types of breast cancer are typically based on which breast cells turn into cancer. The types include ductal carcinoma, which begins in the cells of the ducts and is the most common type; lobular carcinoma, which begins in the lobules and is more often found in both breasts than other types of breast cancer; inflammatory breast cancer, in which cancer cells block lymph vessels in the skin of the breast; Paget's disease of the breast, which is a cancer involving the skin of the nipple. The signs and symptoms of breast cancer include a new lump or thickening in or near the breast or in the armpit; change in the size or shape of the breast; a dimple or puckering in the skin of the breast; a nipple turned inward into the breast; nipple discharge other than breast milk; scaly, red, or swollen skin in the nipple area or the breast; and pain in any area of the breast. According to several National Library of Medicine accepted studies, GABA has significant prognostic value in breast cancer. Breast cancer metastases overexpress many variables related to GABA and are able to proliferate by metabolizing GABA as a biosynthetic energy source, enabling metastatic spread to the brain and cerebrospinal fluid.

Squamous cell carcinoma is the second most common form of skin cancer, characterized by abnormal, accelerated growth of cells that make up the middle and outer layers of the skin. According to the Mayo Clinic and Healthline, squamous cell carcinoma of the skin is usually not life-threatening, though it can be aggressive. Squamous cell carcinoma can also be found in other areas of the body, including the mouth and lungs, but it is much rarer. Most squamous cell carcinomas of the skin result from prolonged exposure to ultraviolet (UV) radiation. Signs and symptoms of squamous cell carcinoma of the skin include a firm, red nodule; a flat sore with a scaly crust; a new sore or raised area on an old scar or ulcer; a rough, scaly patch on the lip that may evolve to an open sore; a red sore or rough patch inside the mouth; and a red, raised patch or wartlike sore on or in the anus or genitals.

Lung cancer originates in the lungs and may spread to lymph nodes or other organs in the body. As many as 40% of people with lung cancer develop brain metastases. According to the Centers for Disease Control and Prevention (CDC), lung cancers are grouped into two main types called small cell and non-small cell (including adenocarcinoma and squamous cell carcinoma). These types of lung cancer grow differently and are treated differently. Non-small cell lung cancer is more common than small cell lung cancer. People who smoke tobacco have the greatest risk of lung cancer. Lung cancer typically doesn't cause signs and symptoms in its earliest stages. Signs and symptoms of lung cancer typically occur when the disease is advanced and may include a new cough that doesn't go away; coughing up blood; shortness of breath; chest pain; hoarseness; losing weight without trying; bone pain; and headache.

Cancer of the peritoneum is cancer that has started in or has spread to the abdominal cavity. According to the NIH, whether the cancer starts in the peritoneum or spreads from somewhere else (i.e., the ovaries, colon, appendix, etc.), it is considered advanced once it is in the peritoneum and can be referred to as peritoneal carcinomatosis. Patients in the early stages will often have few symptoms until the disease is advanced. When symptoms occur, they are often vague and nonspecific, such as fatigue; abdominal swelling; diffuse abdominal pain; frequent urination; bowel changes; abnormal vaginal bleeding; an abdominal mass; unintended weight loss; and a sense of fullness when eating. As the disease progresses, fluid may build up in the abdomen, causing additional abdominal discomfort, nausea and vomiting, and shortness of breath. Complications of peritoneal cancer may include bowel and urinary tract obstruction, sometimes requiring a stent or nephrostomy tube.

Hepatocellular cancer is the most common type of primary liver cancer. According to the Mayo Clinic and the American Cancer Society, hepatocellular carcinoma occurs most often in people with chronic liver diseases, such as cirrhosis caused by hepatitis B or hepatitis C infection, as well as people who drink large amounts of alcohol and who have an accumulation of fat in the liver. Most patients do not demonstrate symptoms in the early stages of primary liver cancer. When symptoms do appear, they may include losing weight without trying; loss of appetite; upper abdominal pain; nausea and vomiting; general weakness and fatigue; abdominal swelling; jaundice; and white, chalky stools. GABA can be a by-product of liver disease and contributes to hepatic encephalopathy in patients with cirrhosis, which can further lead to hepatocellular cancer. Decreases in GABAergic activity may contribute to the pathogenesis of hepatocellular carcinoma.

Gastric cancer can affect any part of the stomach. In most of the world, stomach cancers form in the stomach body. However, according to the Mayo Clinic, in the United States, stomach cancer is more likely to affect the gastroesophageal junction. Although it is not clear what causes gastric cancer, factors that increase the risk include gastroesophageal reflux disease; obesity; a diet high in salty and smoked foods; a diet low in fruits and vegetables; family history; infection with Helicobacter pylori; gastritis; smoking; and stomach polyps. Symptoms of stomach cancer may include difficulty swallowing; feeling bloated after eating; feeling full after eating small amounts; heartburn; indigestion; nausea; stomach pain; unintentional weight loss; and vomiting. GABA has been linked to the proliferation of gastrointestinal cancer types in several studies and has served as a tumor signaling molecule for gastric cancer.

Pancreatic cancer can develop from two kinds of cells in the pancreas: exocrine cells and neuroendocrine cells, such as islet cells. According to the National Cancer Institute, the exocrine type is more common and is usually found at an advanced stage. Pancreatic neuroendocrine tumors, also called islet cell tumors, are less common but have a better prognosis. It's not clear what causes pancreatic cancer, but some factors that may increase the risk include smoking and having inherited gene mutations. Symptoms of pancreatic cancer often do not occur until the disease is advanced and may include abdominal pain that radiates to the back; loss of appetite or unintended weight loss; jaundice; light-colored stools; dark-colored urine; itchy skin; new diagnosis of diabetes or existing diabetes becoming more difficult to control; blood clots; and fatigue. GABA has been linked to stimulation of pancreatic cancer growth.

Glioblastoma, also referred to as a grade IV astrocytoma, is a fast-growing and aggressive brain tumor. It invades the nearby brain tissue, but generally does not spread to distant organs. According to the American Association of Neurological Surgeons, glioblastomas can arise in the brain de novo or evolve from lower-grade astrocytoma. The cause of glioblastomas is unknown, but glioblastomas tend to occur more often in older adults and men. Symptoms may vary based on the location of the tumor but may include persistent headaches; double or blurred vision; vomiting; loss of appetite; changes in mood and personality; changes in ability to think and learn; new onset of seizures; gradual speech difficulty.

Cervical cancer occurs in the lower part of the uterus that connects to the vagina. According to the CDC, various strains of the human papillomavirus, a sexually transmitted infection, play a role in causing most cervical cancer. In a small percentage of people, the virus can survive for years, contributing to the process that causes some cervical cells to become cancer cells. Early-stage cervical cancer generally produces no symptoms. Symptoms of more-advanced cervical cancer include vaginal bleeding after intercourse, between periods, or after menopause; watery, bloody vaginal discharge that may be heavy and have a foul odor; and pelvic pain or pain during intercourse.

Ovarian cancer originates in the ovaries, or in the related areas of the fallopian tubes or the peritoneum. According to the CDC, ovarian cancers exhibit a variety of different tumor types. The most common tumor type is high-grade serous carcinoma, occurring in about 70% of ovarian cancer cases. The cause of ovarian cancer is unknown, but risk factors include family history, genetic mutation of BRCA1 or BRCA 2, endometriosis, and being middle aged or older. Signs and symptoms of ovarian cancer may include abdominal bloating or swelling; weight loss; discomfort in the pelvic area; fatigue; back pain; changes in bowel habits; and frequent urination. Ovarian cancer has been associated with GABA and it may be used for detection.

Bladder cancer most often begins in the urothelial cells that line the bladder, ureters, and kidneys. According to the American Cancer Society, older adults are at a higher risk for bladder cancer. Most bladder cancers are diagnosed at an early stage when the cancer is highly treatable, but bladder cancer has a high reoccurrence rate. Symptoms of bladder cancer include blood in the urine; frequent urination; painful urination; and back pain. GABA has been associated with the promotion and detection of bladder cancer in several studies.

Colorectal cancer originates in the colon or rectum and can be called either colon cancer or rectal cancer, depending on the source. According to the American Cancer Society, most colorectal cancers start as a polyp on the inner lining of the colon or rectum. Not all polyps become cancerous, but depending on the type of polyp it is, the risk can be higher or lower. Hyperplastic polyps and inflammatory polyps are very low risk. Adenomatous polyps, sessile serrated polyps, and traditional serrated adenomas are high risk and referred to as “pre-cancerous.” Symptoms of colon cancer include a persistent change in bowel habits, including diarrhea or constipation, or a change in the consistency of the stool; rectal bleeding or blood in stool; persistent abdominal discomfort, such as cramps, gas or pain; weakness or fatigue; and unexplained weight loss. GABA has been associated with the proliferation and detection of colorectal cancer in several NIH sponsored studies.

Endometrial or uterine carcinoma begins in the lining or glands of the uterus. Most cases occur in women over the age of 55. Risk factors for endometrial or uterine cancer include obesity, family history, age, and hormone effectors (e.g., estrogen, birth control, pregnancy, and polycystic ovarian syndrome). According to the Mayo Clinic, a key symptom is abnormal vaginal bleeding, such as bleeding after menopause or bleeding between periods. Other symptoms may include pelvic pain and pain during sex, but some women experience no symptoms at all. Endometrial and uterine cancers have been associated with GABA and have GABA receptors that facilitate cancer proliferation and can be used for detection of abnormalities.

Salivary gland cancer begins in one of the salivary glands, including the parotid glands, submandibular glands, and sublingual glands. According to the Mayo Clinic, most salivary gland tumors occur in the parotid glands. Most salivary gland tumors are benign, but sometimes they can be cancerous. Symptoms of a salivary gland tumor may include a lump or swelling on or near the jaw or in the neck or mouth; numbness in the face; muscle weakness on one side of the face; persistent pain in the area of a salivary gland; difficulty swallowing; and trouble opening the mouth widely.

Kidney/renal cancer begins in the lining of tubules in the kidney. According to the Mayo Clinic, the incidence of kidney cancer seems to be increasing, which is most likely due to technological improvements in testing and imaging. Risk factors for renal cancer include smoking, abusing pain medications, obesity, family history, and high blood pressure. Kidney cancer can occur at any age, but young children are more likely to develop a kind of kidney cancer called Wilms' tumor. Kidney cancer is often discovered at an early stage, when the cancer is small, confined to the kidney, and usually does not have symptoms. Later stage symptoms may develop, including blood in urine; pain in back or side that persists; loss of appetite; unexplained weight loss; tiredness; and fever. GABA has emerged as a tumor signaling molecule that controls proliferation in renal cancers and can be used for detecting its presence.

Prostate cancer occurs in the male prostate and is the most common cancer among men in the United States. According to the American Cancer Society, prostate cancer usually grows very slowly and finding and treating it before symptoms occur may not improve the patient's health or help them live longer. Prostate cancer is rare in men under 40, but the risk of developing prostate cancer rises rapidly after age 50. Additionally, inherited mutations of the BRCA1 or BRCA2 genes can increase risk. Prostate cancer may cause no signs or symptoms in its early stages, but more advanced stages may cause symptoms such as: trouble urinating; decreased force in the stream of urine; blood in the urine; blood in the semen; bone pain; losing weight without trying; and erectile dysfunction. GABA has been reported to increase cellular proliferation associated with prostate cancers.

Vulvar cancer is a rare cancer that occurs on the outer surface of female genitalia, including the labia and clitoris. Though it can occur at any age, it is most common in older adults. According to the NIH, persistent infection of the human papillomavirus can increase risk of vulvar cancer, as may smoking. Symptoms of vulvar cancer may include persistent itching; localized pain and tenderness; bleeding that is not from menstruation; skin changes, such as color changes or thickening; and lumps, wartlike bumps, or open sores.

Thyroid cancer originates in the thyroid gland. The two most common types of thyroid cancers are papillary and follicular. Papillary thyroid cancer is the most common type and can occur at any age. Papillary thyroid cancer tends to grow slowly and often spreads to lymph nodes in the neck. Follicular cancer is less common and can spread through the blood to distant organs, particularly the lungs and bones. According to the American Thyroid Association, factors that may increase risk for thyroid cancer include history of exposure to high doses of radiation, family history, and being above the age of 40. Most thyroid cancers do not cause any symptoms in early stages, but as thyroid cancer grows, it may cause a nodule that can be felt through the skin on the neck; changes of the voice, including increasing hoarseness; difficulty swallowing; swollen lymph nodes in the neck pain in the neck and throat. GABA alterations have also demonstrated effects on proliferation of thyroid cancers.

Head and neck cancers usually begin in the squamous cells that line the mucosal surfaces of the head and neck, such as inside the oral cavity, throat, and the larynx. Less common head and neck cancers include paranasal sinus and nasal cavity cancers as well as salivary gland cancers. Alcohol and tobacco are major risk factors for cancers of the head and neck. Cancers of the oropharynx are also strongly linked to human papillomavirus. UV light can contribute to risk of cancer on the lips. An infection with the Epstein-Barr virus can raise the risk of cancers in the nose, behind the nose, and cancers of the salivary glands. Chemical and radiation exposures, including wood dust, formaldehyde, asbestos, nickel, etc., can increase the risk of nasopharynx cancers. Symptoms can vary based on the type of cancer but can include open sores that persist; unusual pain or bleeding; trouble with chewing, swallowing, and speaking; and localized swelling. GABA has demonstrated a proliferating effect on some head and neck cancers, including head and neck squamous cell carcinoma.

B-Cell lymphoma is a type of non-Hodgkin lymphoma that originates in the B-cells. There are many subtypes that fall in this category. According to the American Cancer Society, diffuse large B-cell lymphoma (DLBCL) and follicular lymphoma the most common subtypes. DLBCL is the most common type and occurs mostly in older adults. DLBCL tends to be aggressive and fast-growing. Follicular lymphoma is less common and less aggressive and is very rare in young people. Symptoms of non-Hodgkin's lymphomas may include swollen lymph nodes in the neck, armpits or groin; abdominal pain or swelling; chest pain, coughing or trouble breathing; persistent fatigue; fever; night sweats; and unexplained weight loss.

Chronic lymphocytic leukemia (CLL) is another subtype of B-Cell non-Hodgkin lymphoma where most of the cancer cells are found in the blood and bone marrow. CLL progresses slowly and usually affects older adults. The exact mechanism that causes CLL is unknown, but age and chemical exposures appear to be risk factors. According to the American Cancer Society, CLL may not cause any symptoms in the early stages, but when symptoms do occur, they may include swollen lymph nodes; fatigue; fever; pain due to an enlarged spleen; night sweats; weight loss; frequent infections; and easy bruising.

Acute lymphoblastic leukemia (ALL) is a type of cancer of the blood and bone marrow that affects white blood cells. According to the American Cancer Society, ALL is the most common childhood cancer, and it is very aggressive and fast acting. Possible risk factors for ALL include exposure to x-rays before birth; exposure to radiation; certain changes in chromosomes or genes; and certain genetic conditions, such as down syndrome; neurofibromatosis type 1; bloom syndrome; and constitutional mismatch repair deficiency. Symptoms may include enlarged lymph nodes; bruising; fever; bone and joint pain; bleeding from the gums; shortness of breath; fatigue and weakness; loss of appetite; and frequent infections. GABA is involved in the proliferation and spread of ALL and has been observed in several studies.

Hairy cell leukemia (HCL) is another, very rare, subtype of B-Cell non-Hodgkin lymphoma. The affected cells are B lymphocytes found in the bone marrow, spleen, and blood with projections that give a “hairy” appearance. According to the Mayo Clinic, HCL affects more men than women, and it occurs most commonly in middle-aged or older adults. HCL is considered a chronic disease because it may never completely disappear, although treatment can lead to a remission for years. Some people have no signs or symptoms of hairy cell leukemia, but a blood test for another disease or condition may inadvertently reveal hairy cell leukemia. When symptoms do occur, they may include a feeling of fullness in the abdomen that makes it uncomfortable to eat more than a little at a time; fatigue; easy bruising; recurring infections; weakness; and unintentional weight loss.

Chronic myeloid leukemia (CML) is an uncommon type of cancer that originates in certain blood-forming cells of the bone marrow. CML is a slow growing leukemia, but it can change into a fast-growing acute leukemia that is difficult to treat. CML occurs mostly in adults, but very rarely it occurs in children, too. According to the Mayo Clinic, CML often does not cause symptoms and might be detected during a blood test. When symptoms occur, they may include bone pain; easy bleeding; feeling full after eating a small amount of food; fatigue; fever; unintentional weight loss; loss of appetite; pain or fullness below the ribs on the left side; and night sweats.

Acute myeloid leukemia (AML) is a cancer of the blood and bone marrow. It is the most common type of acute leukemia in adults and is very aggressive and progresses rapidly. According to the Mayo Clinic, males and adults over the age of 65 are at higher risk of AML, as well as smokers, those with prior exposure to radiation and dangerous chemicals, and those with other blood disorders. Symptoms of early-stage AML may mimic those of the flu or other common diseases. Later stages may have symptoms including fever; bone pain; lethargy and fatigue; shortness of breath; pale skin; frequent infections; easy bruising; and unusual bleeding, such as frequent nosebleeds and bleeding from the gums.

Melanoma is the most dangerous type of skin cancer. It grows quickly and can spread to any organ. Melanoma originates in melanocytes which produce melanin. According to National Cancer Institute, the exact cause of melanomas is not clear, but exposure to ultraviolet radiation from sunlight or tanning lamps/beds increases the risk of developing melanoma. The risk of melanoma seems to be increasing in people under 40, especially women. The first melanoma symptoms often are a change in an existing mole or the development of a new pigmented or unusual-looking growth on the skin. Malignant moles vary greatly in appearance. Some signs that a pigmentation may be malignant are if the growth is asymmetrical in shape; has an irregular border; changes in color; has a growth in diameter; or appears to be evolving in color or shape. Melanomas and GABA have demonstrated a direct relationship and melanomas have GABA receptors that proliferate cancerous growth.

Testicular cancer occurs in the testes and is the most common cancer in American males between ages 15 and 35. There are two primary forms: seminoma and non-seminoma. Seminoma is slow-growing and primarily affects people in ages 40-60. Non-seminoma grows more rapidly and affects mainly people in their teens, 20s, and 30s. Symptoms of testicular cancer include a lump or enlargement in either testicle; a feeling of heaviness in the scrotum; a dull ache in the abdomen or groin; a sudden collection of fluid in the scrotum; pain or discomfort in a testicle or the scrotum; enlargement or tenderness of the breasts; and back pain. Testicular cancer usually affects only one testicle. Testicular cancer and GABA have demonstrated a direct relationship and wherein GABA proliferates cancerous cell growth and sperm motility changes.

Brain metastases (metastatic brain tumors) and leptomeningeal metastasis happen when cancer spreads from an origination or primary cancer site. Brain metastases occur when cancer cells travel to the brain itself. Leptomeningeal metastasis (also called leptomeningeal cancer, leptomeningeal carcinomatosis, leptomeningeal disease (LMD), neoplastic meningitis, meningeal metastasis and meningeal carcinomatosis) is a rare complication of cancer in which the disease spreads from the original tumor site to the meninges surrounding the brain and spinal cord. Leptomeninges are the two innermost layers of tissue that cover the brain and spinal cord. Leptomeningeal metastasis occurs when the cancer cells invade the cerebrospinal fluid and spread throughout the central nervous system. The prognosis is generally poor with survival typically measured in months. The most common cancers to include the leptomeninges are breast cancer, lung cancer, and melanomas because they can metastasize to the subarachnoid space. The condition can also arise from primary brain tumor like medulloblastoma or glioma. The most common symptoms of leptomeningeal cancer are pain and seizures. The other symptoms may include headaches (usually associated with nausea, vomiting, light-headedness), gait difficulties from weakness or ataxia, memory problems, incontinence, sensory abnormalities. In some cases, symptoms may include double vision, numb chin, back pain, leg weakness, sphincter-related problems, hydrocephalus, loss of urine control, difficulty walking, limb weakness and paresthesia, bowel and bladder dysfunction, double vision, trigeminal sensory or motor loss, cochlear dysfunction, confusion and cognitive impairment.

Gamma-aminobutyric acid (GABA) is metabolized by transamination by gamma aminobutyric acid aminotransferase (GABA-AT) (also known as GABA-transaminase or ABAT). Inhibition of this enzymatic process reduces the degradation of GABA, leading to an increase in the concentration of extracellular and neuronal GABA. Vigabatrin is a GAB A-AT inhibitor that has been used to treat treatment resistant epilepsy and infantile spasms. It has been suggested that the inhibitory effect of vigabatrin on IKCa channels could be an important underlying mechanism of vigabatrin-induced antineoplastic actions associated with glioma. Hung et al., BMC Pharmacol Toxicol 22, 6 (2021). Vigabatrin has been associated with some potentially serious side effects. Its use has been limited due to potential retinal toxicity and subsequent visual field defects. (1S,3S)-3-amino+difluoromethylenyl-1-cyclopentanoic acid (also known as CPP-115) is a GABA-AT inhibitor which is 186 times more efficient in inactivating GABA-AT than vigabatrin. Preclinical data for CPP-115 has been reported to show that significantly lower drug dosages afford comparable pharmacokinetics, improved tolerability, and a more favorable toxicity profile when compared with vigabatrin. See, Prescot et al., Neuropsychopharmacology (2018) 43, 646-654. See also, U.S. Pat. No. 9,993,449, incorporated herein by reference. (S)-3-amino-4-(difluoromethylenyl)cyclopent-1-ene-1-carboxylic acid (also known as OV329) is a GABA-AT inhibitor that has been shown to be 9.8 times more efficient as an inactivator of GABA-AT than CPP-115. Id. U.S. Pat. Nos. 9,603,820 and 9,993,449 describe use of CPP-115 and OV329, respectively, for treatment of hepatocellular carcinoma.

Many cancers are treated with surgery, chemotherapy, radiation, or combinations thereof. Chemotherapeutic agents used in the treatment of cancer are known to produce serious and unpleasant side effects in patients. For example, some chemotherapeutic agents can cause neuropathy, nephrotoxicity (e.g., hyperlipidemia, proteinuria, hypoproteinemia, combinations thereof, or the like), stomatitis, mucositisemesis, alopecia, anorexia, esophagitis amenorrhea, decreased immunity, anemia, high tone hearing loss, cardiotoxicity, fatigue, neuropathy, myelosuppression, to name a few. In some instances, chemotherapy is not effective or loses effectiveness after a period of efficacy, either during treatment, or shortly after the treatment regimen concludes (i.e., the treatment regimen does not result in a cure). Improved methods for the treatment of cancer, and compositions capable of delivering bioactive agents to aid in the treatment of cancer remain desirable. Moreover, in view of the high levels of mortality attributable to cancer, there exists a need for additional treatments to combat the disease.

### SUMMARY

Methods and compositions for treating cancer are provided. In embodiments, methods for treating cancer include administering an effective amount of (S)-3-amino-4-(difluoromethylenyl)cyclopent-1-ene-1-carboxylic acid or a pharmaceutically acceptable salt thereof to a subject in need thereof. In embodiments, compositions for treating cancer which include (S)-3-amino-4-(difluoromethylenyl)cyclopent-1-ene-1-carboxylic acid or a pharmaceutically acceptable salt thereof are administered to a subject in need thereof. In embodiments, the cancer is medulloblastoma, glioma, breast cancer, squamous cell cancer, melanoma, lung cancer, cancer of the peritoneum, hepatocellular cancer, gastric cancer, pancreatic cancer, glioblastoma, cervical cancer, ovarian cancer, testicular cancer, bladder cancer, colorectal cancer, endometrial or uterine carcinoma, salivary gland carcinoma, kidney or renal cancer, prostate cancer, vulval cancer, thyroid cancer, head and neck cancer, B-cell lymphoma, chronic lymphocytic leukemia (CLL), acute lymphoblastic leukemia (ALL), hairy cell leukemia, leptomeningeal carcinomatosis, or chronic myeloblastic leukemia and subtypes thereof. In embodiments, (S)-3-amino-4-(difluoromethylenyl)cyclopent-1-ene-1-carboxylic acid or a pharmaceutically acceptable salt thereof reduces metastasis of the foregoing cancers. In embodiments, (S)-3-amino-4-(difluoromethylenyl)cyclopent-1-ene-1-carboxylic acid or a pharmaceutically acceptable salt thereof prevents metastasis of the foregoing cancers. In embodiments, an effective amount of (S)-3-amino-4-(difluoromethylenyl)cyclopent-1-ene-1-carboxylic acid or a pharmaceutically acceptable salt thereof is between about 0.0001 mg/kg to about 30 mg/kg. In embodiments, 0.5 mg/kg to 6.5 mg/kg of (S)-3-amino-4-(difluoromethylenyl)cyclopent-1-ene-1-carboxylic acid or a pharmaceutically acceptable salt thereof is administered to a subject in need thereof. In embodiments, an effective amount of (S)-3-amino-4-(difluoromethylenyl)cyclopent-1-ene-1-carboxylic acid or a pharmaceutically acceptable salt thereof is administered to a subject once, twice daily, three times daily or four times daily.

### DETAILED DESCRIPTION

Methods and compositions for treating cancer are provided. In embodiments, methods and compositions for treating cancer are provided that reduce or prevent metastasis without unwanted side effects associated with commonly used chemotherapeutic agents. In embodiments, methods for treating cancer include administering to a subject in need thereof an effective amount of (S)-3-amino-4-(difluoromethylenyl)cyclopent-1-ene-1-carboxylic acid or a pharmaceutically acceptable salt thereof (interchangeably referred to herein as OV329). In embodiments, methods for inhibiting cancer proliferation and metastasis are provided that include administering to a subject in need thereof an effective amount of (S)-3-amino-4-(difluoromethylenyl)cyclopent-1-ene-1-carboxylic acid or a pharmaceutically acceptable salt thereof.

In leptomeningeal metastasis, for metastasis to occur, a series of sequential steps must be achieved by tumor cells. At the primary site, the tumor cells proliferate, invade the surrounding environment, enter the cerebrospinal fluid and spread throughout the central nervous system. The metastatic tumor cells will then be required to survive in the microenvironment of the meninges and cerebrospinal fluid. The meninges and cerebrospinal fluid provide a relatively nutrient poor environment. Metastatic brain cancer cells such as metastatic MB cells use GABA-AT to maintain viability in the metabolite-scarce cerebrospinal fluid by using GABA as an energy source substitute, thereby facilitating leptomeningeal metastasis formation. Martirosian et al., 2021, Cell Reports 35, 109302.

In accordance with the present disclosure, but without wishing to be bound by any theory, by administering (S)-3-amino-4-(difluoromethylenyl)cyclopent-1-ene-1-carboxylic acid or a pharmaceutically acceptable salt thereof, which is a GABA-AT inhibitor, GABA metabolism is blocked or reduced and metastatic MB cells are unable to adequately utilize GABA as an energy source substitute, thereby creating an even more unfavorable nutrient poor environment for metastatic MB cells in the cerebrospinal fluid and throughout the central nervous system. In this manner, metastasis is prevented or reduced. Indeed, any of the cancers described herein utilizing GABA catabolism via GABA-AT as an energy source are vulnerable to the effects of (S)-3-amino-4-(difluoromethylenyl)cyclopent-1-ene-1-carboxylic acid or a pharmaceutically acceptable salt thereof in reducing or preventing utilization of GABA as an energy source, thereby preventing or reducing tumor growth and/or metastasis.

In embodiments, (S)-3-amino-4-(difluoromethylenyl)cyclopent-1-ene-1-carboxylic acid or a pharmaceutically acceptable salt thereof is administered to a subject at low doses that inhibit GABA-AT but do not dramatically influence circulating GABA, which as discussed above, can have a pro-tumor and/or metastasis effect.

Significantly, many anti-cancer compounds fail to cross the blood brain barrier and cannot be utilized to treat brain cancer. (S)-3-amino-4-(difluoromethylenyl)cyclopent-1-ene-1-carboxylic acid or a pharmaceutically acceptable salt thereof can cross the blood brain barrier and treat brain cancer and leptomeningeal metastasis.

The term “cancer”, as used herein refers to a proliferative disorder caused or characterized by a proliferation of cells which have lost susceptibility to normal growth control. Cancers of the same tissue type usually originate in the same tissue and may be divided into different subtypes based on their biological characteristics. Four general categories of cancer are carcinoma (epithelial cell derived), sarcoma (connective tissue or mesodermal derived), leukemia (blood-forming tissue derived) and lymphoma (lymph tissue derived). Non-limiting examples of cancers susceptible to treatment with (S)-3-amino-4-(difluoromethylenyl)cyclopent-1-ene-1-carboxylic acid or a pharmaceutically acceptable salt thereof include medulloblastoma, glioma, breast cancer, squamous cell cancer, melanoma, lung cancer, cancer of the peritoneum, hepatocellular cancer, gastric cancer, pancreatic cancer, glioblastoma, cervical cancer, ovarian cancer, testicular cancer, bladder cancer, colorectal cancer, endometrial or uterine carcinoma, salivary gland carcinoma, kidney or renal cancer, prostate cancer, vulval cancer, thyroid cancer, head and neck cancer, B-cell lymphoma, chronic lymphocytic leukemia, acute lymphoblastic leukemia, hairy cell leukemia, or chronic myeloblastic leukemia and subtypes thereof, including refractory versions of any of the above cancers, or a combination of one or more of the above cancers. The above cancers can utilize GABA-AT to metabolize GABA as a source of energy.

As used herein, the terms “cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass, e.g., by procedures such as CAT scan, MR imaging, X-ray, ultrasound or palpation, and/or which is detectable because of the expression of one or more cancer-specific antigens in a sample obtainable from a patient. Leukemia is clinically detectable using one or more of complete blood counts, pallor, blood smears, and bone marrow smears. Advanced leukemias in certain subjects can manifest solid tumors.

The structure of (S)-3-amino-4-(difluoromethylenyl)cyclopent-1-ene-1-carboxylic acid may be represented as follows:

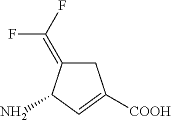 [SMILES: N[C@H]1C=C(C(=O)O)CC1=C(F)F]

In embodiments, (S)-3-amino-4-(difluoromethylenyl)cyclopent-1-ene-1-carboxylic acid may be provided as an acid addition salt, a zwitter ion hydrate, zwitter ion anhydrate, hydrochloride or hydrobromide salt, or in the form of the zwitter ion monohydrate. Acid addition salts, include but are not limited to, maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, pantothenic, p-amino-benzoic, glutamic, benzene sulfonic or theophylline acetic acid addition salts, as well as the 8-halotheophyllines, for example 8-bromo-theophylline. In embodiments, inorganic acid addition salts, including but not limited to, hydrochloric, hydrobromic, hydroiodic, sulfuric, sulfamic, phosphoric or nitric acid addition salts may be used.

As can be seen from the Examples herein, an effective amount of (S)-3-amino-4-(difluoromethylenyl)cyclopent-1-ene-1-carboxylic acid or a pharmaceutically acceptable salt thereof for treatment of cancer herein is surprisingly devoid of or exhibits less unwanted side-effects that are normally associated with GABA-AT inhibitors such as vigabatrin, e.g., retinal toxicity and reduced motor coordination. The term “effective amount” as applied to (S)-3-amino-4-(difluoromethylenyl)cyclopent-1-ene-1-carboxylic acid or a pharmaceutically acceptable salt thereof refers to an amount effective to prevent or treat cancer. An “effective amount” of (S)-3-amino-4-(difluoromethylenyl)cyclopent-1-ene-1-carboxylic acid or a pharmaceutically acceptable salt thereof may be determined by various factors, for example, administration method, target site, the patient's condition, and the like. Therefore, the dosage when used in the human body should be determined in appropriate amounts in consideration of safety and efficacy. It is also possible to estimate the amount used in humans from the effective amount determined by animal experiments. See, e.g., Reagan-Shaw et al., FASEB J. 2008 March; 22(3):659-61.

The term “prevent”, including grammatical variants thereof such as “preventing” and “prevention” as used herein refers to actions that inhibit or delay the development, spread and recurrence of cancer by administration of the (S)-3-amino-4-(difluoromethylenyl)cyclopent-1-ene-1-carboxylic acid or a pharmaceutically acceptable salt thereof. The term “treat”, including grammatical variants thereof such as “treating” and “treatment” as used herein refers to all types of actions that reduce the proliferation of cancer cells, facilitate the death of cancer cells and/or reduce symptoms of cancer by the administration (S)-3-amino-4-(difluoromethylenyl)cyclopent-1-ene-1-carboxylic acid or a pharmaceutically acceptable salt thereof according to the disclosure herein. “Treating cancer” herein includes inhibiting cancer proliferation and metastasis of cancer cells. For example, in embodiments, “treating” may include a reduction in seeding of metatastic cells or a reduction in seeding of tumor cells.

An “effective amount” herein ranges from about 0.0001 mg/kg to about 30 mg/kg of (S)-3-amino-4-(difluoromethylenyl)cyclopent-1-ene-1-carboxylic acid or a pharmaceutically acceptable salt thereof. In embodiments, a relatively low dose of (S)-3-amino-4-(difluoromethylenyl)cyclopent-1-ene-1-carboxylic acid or a pharmaceutically acceptable salt thereof that inhibits GABA-AT without dramatically influencing circulating GABA ranges from about 0.2 mg/kg to 10 mg/kg. In embodiments, a relatively low dose of (S)-3-amino-4-(difluoromethylenyl)cyclopent-1-ene-1-carboxylic acid or a pharmaceutically acceptable salt thereof that inhibits GABA-AT without dramatically influencing circulating GABA ranges from about 0.5 mg/kg to about 6.5 mg/kg. In embodiments, from about 20 mg to about 400 mg of (S)-3-amino-4-(difluoromethylenyl)cyclopent-1-ene-1-carboxylic acid or a pharmaceutically acceptable salt thereof is given once, twice, thrice or four times a day. For example, a pharmaceutical composition including an effective amount of (S)-3-amino-4-(difluoromethylenyl)cyclopent-1-ene-1-carboxylic acid or a pharmaceutically acceptable salt thereof may contain from about 20 mg to about 25 mg, about 25 mg to about 30 mg, about 30 mg to about 35 mg, about 35 mg to about 40 mg, about 40 mg to about 45 mg, about 45 mg to about 50 mg, about 50 mg to about 55 mg, about 55 mg to about 60 mg, about 60 mg to about 65 mg, about 65 mg to about 70 mg, about 70 mg to about 75 mg, about 75 mg to about 80 mg, about 80 mg to about 85 mg, about 85 mg to about 90 mg, about 90 mg to about 95 mg, about 95 mg to about 100 mg, about 100 mg to about 105 mg, about 105 mg to about 110 mg, about 110 mg to about 115 mg, about 115 mg to about 120 mg, about 120 mg to about 125 mg, about 130 mg to about 135 mg, about 140 mg to about 145 mg, about 150 mg to about 155 mg, about 160 mg to about 165 mg, about 170 mg to about 175 mg, about 180 mg to about 185 mg, about 190 mg to about 195 mg, about 200 mg to about 205 mg, about 210 mg to about 210 mg, about 215 mg to about 220 mg, about 225 mg to about 230 mg, about 235 mg to about 240 mg, about 245 mg to about 250 mg, about 255 mg to about 260 mg, about 265 mg to about 270 mg, about 275 mg to about 280 mg, about 285 mg to about 290 mg, about 295 mg to about 300 mg, about 305 mg to about 310 mg, about 315 mg to about 320 mg, about 325 mg to about 330 mg, about 335 mg to about 340 mg, about 345 mg to about 350 mg, about 355 mg to about 360 mg, about 365 mg to about 370 mg, about 375 mg to about 380 mg, about 385 mg to about 390 mg, about 395 mg to about 400 mg, of (S)-3-amino-4-(difluoromethylenyl)cyclopent-1-ene-1-carboxylic acid or a pharmaceutically acceptable salt thereof. In embodiments a pharmaceutical composition containing an effective amount of (S)-3-amino-4-(difluoromethylenyl)cyclopent-1-ene-1-carboxylic acid or a pharmaceutically acceptable salt thereof includes 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, or 400 mg, of (S)-3-amino-4-(difluoromethylenyl)cyclopent-1-ene-1-carboxylic acid or a pharmaceutically acceptable salt thereof.

In embodiments, (S)-3-amino-4-(difluoromethylenyl)cyclopent-1-ene-1-carboxylic acid or a pharmaceutically acceptable salt thereof is administered to a subject at about 25 mg/per day, 30 mg/per day, 35 mg/per day, 40 mg/per day, 45 mg/per day, 50 mg/per day, 60 mg/per day, 65 mg/per day, 70 mg/per day, 75 mg/per day, 80 mg/per day, 85 mg/per day, 90 mg/per day, 95 mg/per day, 100 mg/per day, 105 mg/per day, 110 mg/per day, 115 mg/per day, 120 mg/per day, 125 mg/per day, 130 mg/per day, 135 mg/per day, 140 mg/per day, 145 mg/per day, 150 mg/per day, 155 mg/per day, 160 mg/per day, 165 mg/per day, 170 mg/per day, 175 mg/per day, 180 mg/per day, 185 mg/per day, 190 mg/per day, 195 mg/per day, 200 mg/per day, 205 mg/per day, 210 mg/per day, 215 mg/per day, 220 mg/per day, 225 mg/per day, 230 mg/per day, 235 mg/per day, 240 mg/per day, 245 mg/per day, 250 mg/per day, 255 mg/per day, 260 mg/per day, 265 mg/per day, 270 mg/per day, 275 mg/per day, 280 mg/per day, 285 mg/per day, 290 mg/per day, 295 mg/per day, 300 mg/per day, 305 mg/per day, 310 mg/per day, 315 mg/per day, 320 mg/per day, 325 mg/per day, 330 mg/per day, 335 mg/per day, 340 mg/per day, 345 mg/per day, 350 mg/per day, 355 mg/per day, 360 mg/per day, 365 mg/per day, 370 mg/per day, 375 mg/per day, 380 mg/per day, 385 mg/per day, 390 mg/per day, 395 mg/per day, or 400 mg/per day, in one or more doses. In embodiments, the subject may be started at a low dose and the dosage may be escalated over time.

In embodiments, the step of administering results in at least one of the following: a 10% reduction in size of tumors, at least a 10% reduction in number of cancer metastases, or at least a 10% improvement in clinical signs and symptoms related to cancer. The term “administration” and grammatical variations thereof such as “administer” and “administering”, as used herein means providing a predetermined substance to an individual or a patient by any appropriate method. For example, (S)-3-amino-4-(difluoromethylenyl)cyclopent-1-ene-1-carboxylic acid or a pharmaceutically acceptable salt thereof may be administered enterally, e.g., orally, or parenterally (for example, by applying in injectable formulations intravenously, subcutaneously, intraperitoneally, or topically). The dosage may vary depending on the patient's body weight, age, sex, health condition, diet, administration time, administration method, excretion rate, the severity of the disease and the like. Liquid formulations for oral administration of (S)-3-amino-4-(difluoromethylenyl)cyclopent-1-ene-1-carboxylic acid or a pharmaceutically acceptable salt thereof include suspensions, oral liquids, emulsions, syrups and the like. In addition to water and liquid paraffin which are simple diluents commonly used, various excipients such as wetting agents, sweeteners, flavors, preservatives and the like may be included. Formulations for parenteral administration include sterile aqueous solutions, non-aqueous solvents, suspensions, emulsions, freeze-dried formulations, suppositories, and the like.

In embodiments, (S)-3-amino-4-(difluoromethylenyl)cyclopent-1-ene-1-carboxylic acid or a pharmaceutically acceptable salt thereof is administered to a subject having cancer via a pharmaceutical composition. Pharmaceutical compositions herein may be administered by any device capable of moving the active substance to target cells. Pharmaceutical compositions herein encompass dosage forms. Dosage forms herein encompass unit doses. In embodiments, as discussed below, various dosage forms including conventional formulations and modified release formulations can be administered one or more times daily. In embodiments, (S)-3-amino-4-(difluoromethylenyl) cyclopent-1-ene-1-carboxylic acid or a pharmaceutically acceptable salt thereof is administered to a subject once or twice a day (e.g., morning and/or night), or three times a day (morning, afternoon and night) or four times a day (morning, afternoon, evening and night). Any suitable route of administration may be utilized, e.g., oral, rectal, nasal, pulmonary, vaginal, sublingual, transdermal, subcutaneous, intravenous, intraarterial, intrathecal, intramuscular, intraperitoneal, intratumoral, intracranial and intraventricular routes.

Suitable dosage forms include tablets, capsules, oral liquids, powders, aerosols, transdermal modalities such as topical liquids, patches, creams and ointments, parenteral formulations and suppositories. In embodiments, (S)-3-amino-4-(difluoromethylenyl) cyclopent-1-ene-1-carboxylic acid or a pharmaceutically acceptable salt thereof is used to manufacture a medicament for treatment of cancer or inhibiting cancer proliferation and metastasis. Those skilled in the art are familiar with pharmaceutical compounding techniques for formulating and manufacturing pharmaceutical compositions. See, e.g., E. W. Martin ed., Remington's Pharmaceutical Sciences, 18th ed. (1990), Mack Publishing Co.

In embodiments, methods of treating cancer including inhibiting cancer proliferation and metastasis are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in cancer symptoms for more than 1 hour after administration to the subject. In embodiments, methods of treating cancer including inhibiting cancer proliferation and metastasis are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in one or more cancer symptoms for more than 2 hours after administration to the subject. In embodiments, methods of treating cancer including inhibiting cancer proliferation and metastasis are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in one or more cancer symptoms for more than 3 hours after administration to the subject. In embodiments, methods of treating cancer including inhibiting cancer proliferation and metastasis are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in one or more cancer symptoms for more than 4 hours after administration to the subject. In embodiments, methods of treating cancer including inhibiting cancer proliferation and metastasis are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in one or more cancer symptoms for more than 6 hours after administration to the subject. In embodiments, methods of treating cancer including inhibiting cancer proliferation and metastasis are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in one or more cancer symptoms for more than 8, 10, 12, 14, 16, 18, 20, 22 or 24 hours after administration to the subject. In embodiments, the pharmaceutical compositions provide improvement of next day functioning of the subject. For example, the pharmaceutical compositions may provide improvement in one or more cancer symptoms for more than about, e.g., 2 hours, 4 hours, 6 hours, 8 hours, 10 hours, 12 hours, 14 hours, 16 hours, 18 hours, 20 hours, 22 hours or 24 hours after administration and waking from a night of sleep.

In embodiments, pharmaceutical compositions herein may be provided with conventional release or modified release profiles. Pharmaceutical compositions may be prepared using a pharmaceutically acceptable “carrier” composed of materials that are considered safe and effective. The “carrier” includes all components present in the pharmaceutical formulation other than the active ingredient or ingredients. The term “carrier” includes, but is not limited to, diluents, binders, lubricants, disintegrants, fillers, and coating compositions. Those with skill in the art are familiar with such pharmaceutical carriers and methods of compounding pharmaceutical compositions using such carriers. In embodiments, pharmaceutical compositions herein may include a pharmaceutically acceptable additive, which is exemplified by starch, gelatinized starch, microcrystalline cellulose, milk sugar, povidone, colloidal silicon dioxide, calcium hydrogen phosphate, lactose, mannitol, Arabia rubber, pregelatinized starch, corn starch, cellulose powder, hydroxypropyl cellulose, Opadry, sodium starch glycolate, carnauba wax, synthetic aluminum silicate, stearic acid, magnesium stearate, aluminum stearate, calcium stearate, white sugar, dextrose, sorbitol, talc, etc. In embodiments, a pharmaceutically acceptable additive can be added to the composition in an amount of 0.1 to 90 parts by weight but is not limited thereto.

In embodiments, pharmaceutical compositions herein are modified release dosage forms which provide modified release profiles. Modified release profiles may exhibit immediate release, delayed release, or extended release profiles. Conventional (or unmodified) release oral dosage forms such as tablets, capsules, suppositories, syrups, solutions and suspensions typically release medications into the mouth, stomach or intestines as the tablet, capsule shell or suppository dissolves, or, in the case of syrups, solutions and suspensions, when they are swallowed. The pattern of drug release from modified release (MR) dosage forms is deliberately changed from that of a conventional dosage form to achieve a desired therapeutic objective and/or better patient compliance. Types of MR drug products include orally disintegrating dosage forms (ODDFs) which provide immediate release, extended release dosage forms, delayed release dosage forms (e.g., enteric coated), and pulsatile release dosage forms.

An ODDF is a solid dosage form containing a medicinal substance or active ingredient which disintegrates rapidly, usually within a matter of seconds when placed upon the tongue. The disintegration time for ODDFs generally range from one or two seconds to about a minute. ODDFs are designed to disintegrate or dissolve rapidly on contact with saliva. This mode of administration can be beneficial to people who may have problems swallowing tablets whether it be from physical infirmity or psychiatric in nature. Subjects in pain may exhibit such behavior. ODDF's can provide rapid delivery of medication to the blood stream through mucosa resulting in a rapid onset of action. Examples of ODDFs include orally disintegrating tablets, capsules and rapidly dissolving films and wafers.

Extended release dosage forms (ERDFs) have extended release profiles and are those that allow a reduction in dosing frequency as compared to that presented by a conventional dosage form, e.g., a solution or unmodified release dosage form. ERDFs provide a sustained duration of action of a drug. Suitable formulations which provide extended release profiles are well-known in the art. For example, coated slow release beads or granules (“beads” and “granules” are used interchangeably herein) in which (S)-3-amino-4-(difluoromethylenyl)cyclopent-1-ene-1-carboxylic acid or a pharmaceutically acceptable salt thereof is applied to beads, e.g., confectioners nonpareil beads, and then coated with conventional release retarding materials such as waxes, enteric coatings and the like. In embodiments, beads can be formed in which (S)-3-amino-4-(difluoromethylenyl)cyclopent-1-ene-1-carboxylic acid or a pharmaceutically acceptable salt thereof is mixed with a material to provide a mass from which the drug leaches out. In embodiments, the beads may be engineered to provide different rates of release by varying characteristics of the coating or mass, e.g., thickness, porosity, using different materials, etc. Beads having different rates of release may be combined into a single dosage form to provide variable or continuous release. The beads can be contained in capsules or compressed into tablets.

In embodiments, modified dosage forms herein incorporate delayed release dosage forms having delayed release profiles. Delayed release dosage forms can include delayed release tablets or delayed release capsules. A delayed release tablet is a solid dosage form which releases a drug (or drugs) such as (S)-3-amino-4-(difluoromethylenyl)cyclopent-1-ene-1-carboxylic acid or a pharmaceutically acceptable salt thereof at a time other than promptly after administration. A delayed release capsule is a solid dosage form in which the drug is enclosed within either a hard or soft soluble container made from a suitable form of gelatin, and which releases a drug (or drugs) at a time other than promptly after administration. For example, enteric-coated tablets, capsules, particles and beads are well-known examples of delayed release dosage forms. Enteric coated tablets, capsules and particles and beads pass through the stomach and release the drug in the intestine. In embodiments, a delayed release tablet is a solid dosage form containing a conglomerate of medicinal particles that releases a drug (or drugs) at a time other than promptly after administration. In embodiments, the conglomerate of medicinal particles is covered with a coating which delays release of the drug. In embodiments, a delayed release capsule is a solid dosage form containing a conglomerate of medicinal particles that releases a drug (or drugs) at a time other than promptly after administration. In embodiments, the conglomerate of medicinal particles is covered with a coating which delays release of the drug.

Delayed release dosage forms are known to those skilled in the art. For example, coated delayed release beads or granules in which (S)-3-amino-4-(difluoromethylenyl)cyclopent-1-ene-1-carboxylic acid or a pharmaceutically acceptable salt thereof is applied to beads, e.g., confectioners nonpareil beads, and then coated with conventional release delaying materials such as waxes, enteric coatings and the like. In embodiments, beads can be formed in which (S)-3-amino-4-(difluoromethylenyl)cyclopent-1-ene-1-carboxylic acid or a pharmaceutically acceptable salt thereof is mixed with a material to provide a mass from which the drug leaches out. In embodiments, the beads may be engineered to provide different rates of release by varying characteristics of the coating or mass, e.g., thickness, porosity, using different materials, etc. In embodiments, enteric coated granules of (S)-3-amino-4-(difluoromethylenyl)cyclopent-1-ene-1-carboxylic acid or a pharmaceutically acceptable salt thereof can be contained in an enterically coated capsule or tablet which releases the granules in the small intestine. In embodiments, the granules have a coating which remains intact until the coated granules reach at least the ileum and thereafter provide a delayed release of the drug in the colon. Suitable enteric coating materials are well known in the art, e.g., Eudragit® coatings such methacrylic acid and methyl methacrylate polymers and others. The granules can be contained in capsules or compressed into tablets.

In embodiments, (S)-3-amino-4-(difluoromethylenyl)cyclopent-1-ene-1-carboxylic acid or a pharmaceutically acceptable salt thereof is incorporated into porous inert carriers that provide delayed release profiles. In embodiments, the porous inert carriers incorporate channels or passages from which the drug diffuses into surrounding fluids. In embodiments, (S)-3-amino-4-(difluoromethylenyl)cyclopent-1-ene-1-carboxylic acid or a pharmaceutically acceptable salt thereof is incorporated into an ion-exchange resin to provide a delayed release profile. Delayed action may result from a predetermined rate of release of the drug from the resin when the drug-resin complex contacts gastrointestinal fluids and the ionic constituents dissolved therein. In embodiments, membranes are utilized to control rate of release from drug containing reservoirs. In embodiments, liquid preparations may also be utilized to provide a delayed release profile. For example, a liquid preparation consisting of solid particles dispersed throughout a liquid phase in which the particles are not soluble. The suspension is formulated to allow at least a reduction in dosing frequency as compared to that drug presented as a conventional dosage form (e.g., as a solution or a prompt drug-releasing, conventional solid dosage form). For example, a suspension of ion-exchange resin constituents or microbeads.

In embodiments, (S)-3-amino-4-(difluoromethylenyl)cyclopent-1-ene-1-carboxylic acid or a pharmaceutically acceptable salt thereof is incorporated into polymeric delivery vehicles such as those manufactured from cellulose, agarose, polymethacrylate, polystyrene, and polyacrylamide and composites of the foregoing.

In embodiments, (S)-3-amino-4-(difluoromethylenyl)cyclopent-1-ene-1-carboxylic acid or a pharmaceutically acceptable salt thereof is incorporated into a hydrogel-based delivery vehicle. Examples of suitable hydrogels include polyvinyl alcohol, sodium alginate, chitosan, polyvinyl pyrrolidone, derivatives of polyacrylic and/or polymethacrylic acid, and composites of the foregoing.

In embodiments, pharmaceutical compositions described herein are suitable for parenteral administration, including, e.g., intramuscular (i.m.), intravenous (i.v.), subcutaneous (s.c.), intraperitoneal (i.p.), or intrathecal (i.t.). Parenteral compositions must be sterile for administration by injection, infusion or implantation into the body and may be packaged in either single-dose or multi-dose containers. In embodiments, liquid pharmaceutical compositions for parenteral administration to a subject include an active substance, e.g., (S)-3-amino-4-(difluoromethylenyl)cyclopent-1-ene-1-carboxylic acid or a pharmaceutically acceptable salt thereof in any of the respective amounts described above. In embodiments, the pharmaceutical compositions for parenteral administration are formulated as a total volume of about, e.g., 10 ml, 20 ml, 25 ml, 50 ml, 100 ml, 200 ml, 250 ml, or 500 ml. In embodiments, the compositions are contained in a bag, a glass vial, a plastic vial, or a bottle.

Pharmaceutical compositions for parenteral administration provided herein may include one or more excipients, e.g., solvents, solubility enhancers, suspending agents, buffering agents, isotonicity agents, stabilizers or antimicrobial preservatives. When used, the excipients of the parenteral compositions will not adversely affect the stability, bioavailability, safety, and/or efficacy of (S)-3-amino-4-(difluoromethylenyl)cyclopent-1-ene-1-carboxylic acid or a pharmaceutically acceptable salt thereof used in the composition. Thus, parenteral compositions are provided wherein there is no incompatibility between any of the components of the dosage form.

In embodiments, parenteral compositions (S)-3-amino-4-(difluoromethylenyl)cyclopent-1-ene-1-carboxylic acid or a pharmaceutically acceptable salt thereof include a stabilizing amount of at least one excipient. For example, excipients may be selected from the group consisting of buffering agents, solubilizing agents, tonicity agents, antioxidants, chelating agents, antimicrobial agents, and preservative. One skilled in the art will appreciate that an excipient may have more than one function and be classified in one or more defined group.

In embodiments, parenteral compositions (S)-3-amino-4-(difluoromethylenyl)cyclopent-1-ene-1-carboxylic acid or a pharmaceutically acceptable salt thereof and an excipient wherein the excipient is present at a weight percent (w/v) of less than about, e.g., 10%, 5%, 2.5%, 1%, or 0.5%. In embodiments, the excipient is present at a weight percent between about, e.g., 1.0% to 10%, 10% to 25%, 15% to 35%, 0.5% to 5%, 0.001% to 1%, 0.01% to 1%, 0.1% to 1%, or 0.5% to 1%. In embodiments, the excipient is present at a weight percent between about, e.g., 0.001% to 1%, 0.01% to 1%, 1.0% to 5%, 10% to 15%, or 1% to 15%.

In embodiments, parenteral compositions of an active substance, e.g., (S)-3-amino-4-(difluoromethylenyl)cyclopent-1-ene-1-carboxylic acid or a pharmaceutically acceptable salt thereof are provided, wherein the pH of the composition is between about 4.0 to about 8.0. In embodiments, the pH of the compositions is between, e.g., about 5.0 to about 8.0, about 6.0 to about 8.0, about 6.5 to about 8.0. In embodiments, the pH of the compositions is between, e.g., about 6.5 to about 7.5, about 7.0 to about 7.8, about 7.2 to about 7.8, or about 7.3 to about 7.6. In embodiments, the pH of the aqueous solution is, e.g., about 6.8, about 7.0, about 7.2, about 7.4, about 7.6, about 7.7, about 7.8, about 8.0, about 8.2, about 8.4, or about 8.6.

It should be understood that the dosage amounts of (S)-3-amino-4-(difluoromethylenyl)cyclopent-1-ene-1-carboxylic acid, or a pharmaceutically acceptable salt thereof that are provided herein are applicable to all the dosage forms described herein including conventional dosage forms, modified dosage forms, as well as the parenteral formulations described herein. Those skilled in the art will determine appropriate amounts depending on criteria such as dosage form, route of administration, subject tolerance, efficacy, therapeutic goal and therapeutic benefit, among other pharmaceutically acceptable criteria.

In embodiments, (S)-3-amino-4-(difluoromethylenyl)cyclopent-1-ene-1-carboxylic acid or a pharmaceutically acceptable salt thereof may be administered alone, or in combination therapies wherein the subject is also undergoing one or more cancer therapies such as surgery, chemotherapy, radiotherapy, thermotherapy, immunotherapy, hormone therapy and/or laser therapy.

In embodiments, combination therapy can include, e.g., one or more of chemotherapeutics, targeting agents such as antibodies; kinase inhibitors; hormonal agents and the like. Combination therapies can also include conventional therapy, including, but not limited to, antibody administration, vaccine administration, administration of cytotoxic agents, natural amino acid polypeptides, nucleic acids, nucleotide analogues, and biologic response modifiers. Two or more combined compounds may be used together or sequentially. For example, anti-cancer agents that are well known in the art and can be used as a treatment in combination with the (S)-3-amino-4-(difluoromethylenyl)cyclopent-1-ene-1-carboxylic acid or a pharmaceutically acceptable salt thereof. Examples of chemotherapeutic agents include alkylating agents, antimetabolites, natural products, hormones and antagonists, and miscellaneous agents. Examples of alkylating agents include nitrogen mustards such as mechlorethamine, cyclophosphamide, ifosfamide, melphalan (L-sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, 5FU), floxuridine (fluorodeoxyuridine, FUdR), cytarabine (cytosine arabinoside) and gemcitabine; purine analogs such as mercaptopurine (6-niercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel (Abraxane) and docetaxel (Taxotere); epipodophyllotoxins such as etoposide and teniposide; camptothecins such as topotecan and irinotecan; antibiotics such as dactinomycin (actinomycin D), daunorubicin (daunomycin, rubidomycin), doxorubicin, bleomycin, mitomycin (mitomycin C), idarubicin, epirubicin; enzymes such as L-asparaginase; and biological response modifiers such as interferon alpha and interlelukin 2. Examples of hormones and antagonists include luteinising releasing hormone agonists such as buserelin; adrenocorticosteroids such as prednisone and related preparations; progestins such as hydroxyprogesterone caproate, medroxyprogesterone acetate and megestrol acetate; estrogens such as diethylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Examples of miscellaneous agents include thalidomide; platinum coordination complexes such as cisplatin, oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane and aminoglutethimide; RXR agonists such as bexarotene; and tyrosine kinase inhibitors such as imatinib. In some embodiments, the additional cancer therapy is bortezomib administration.

Unless defined otherwise, all technical and scientific terms used herein have the same meanings as commonly understood by one of skill in the art to which the disclosure herein belongs.

The term “about” or “approximately” as used herein means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and/or up to 1% of a given value.

“Improvement” refers to the treatment of cancer including all forms of cancer and means a change for better. “Improvement” may be subjective or objective.

“Improvement in next day functioning” or “wherein there is improvement in next day functioning” refers to improvement after waking from an overnight sleep period wherein the beneficial effect of administration of (S)-3-amino-4-(difluoromethylenyl)cyclopent-1-ene-1-carboxylic acid or a pharmaceutically acceptable salt thereof applies to cancer symptoms and is discernable, either subjectively by a subject or objectively by an observer, for a period of time, e.g., 2 hours, 3 hours, 4 hours, 5 hours, 6 hours, 12 hours, 24 hours, etc. after waking.

“PK” refers to the pharmacokinetic profile. Cₘₐₓ is defined as the highest plasma drug concentration estimated during an experiment (ng/ml). Tₘₐₓ is defined as the time when Cₘₐₓ is estimated (min). AU_{0-∞} is the total area under the plasma drug concentration-time curve, from drug administration until the drug is eliminated (ng·hr/ml or μg·hr/ml). The area under the curve is governed by clearance. Clearance is defined as the volume of blood or plasma that is totally cleared of its content of drug per unit time (ml/min).

“Treating”, “treatment” or “treat” can refer to the following: reducing, improving, relieving, ameliorating, mitigating, inhibiting, reversing and/or alleviating cancer or symptoms of cancer in a subject. In embodiments, “treating”, “treat” or “treatment” may refer to preventing the appearance of clinical symptoms of a disease or condition in a subject that may be afflicted with or predisposed to the disease or condition but does not yet experience or display clinical or subclinical symptoms of the disease or condition. The benefit to a subject to be treated may be statistically significant, mathematically significant, or at least perceptible to the subject and/or the health care practitioner. Nonetheless, prophylactic (preventive) and therapeutic (curative) treatment are two separate embodiments of the disclosure herein.

“Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In embodiments, this term refers to molecular entities and compositions approved by a regulatory agency of the federal or a state government, as the GRAS list under section 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

“Co-administered with”, “administered in combination with”, “a combination of” or “administered along with” may be used interchangeably and mean that two or more agents are administered in the course of therapy. The agents may be administered together at the same time or separately in spaced apart intervals. The agents may be administered in a single dosage form or in separate dosage forms.

“Subject in need thereof” includes individuals that have been diagnosed with cancer or are at risk of developing cancer with reasonable certainty. The methods and compositions including (S)-3-amino-4-(difluoromethylenyl)cyclopent-1-ene-1-carboxylic acid or a pharmaceutically acceptable salt thereof may be provided to any individual including, e.g., wherein the subject is a neonate, infant, a pediatric subject (6 months to 12 years), an adolescent subject (age 12-18 years) or an adult (over 18 years). Subjects include mammals such as humans. “Patient” and “subject” may be used interchangeably herein.

The term “pharmaceutically acceptable salt”, as used herein, refers to derivatives of the compounds defined herein, wherein the parent compound is modified by making acid or base salts thereof. Examples of pharmaceutically acceptable salts include but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include but are not limited to those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic salts. The pharmaceutically acceptable salts can be synthesized from the parent compound, which contains a basic or acidic moiety, by conventional chemical methods.

### EXAMPLES

The examples provided herein are included solely for augmenting the disclosure herein and should not be considered to be limiting in any respect.

### Example 1

### Safety Assessment for (S)-3-Amino-4-(difluoromethylenyl)cyclopent-1-ene-1-carboxylic acid

A non-GLP safety assessment for changes in retinal function and structure were evaluated in rats. Initial results indicate that (S)-3-amino-4-(difluoromethylenyl)cyclopent-1-ene-1-carboxylic acid is well tolerated up to 3 mg/kg/day for 45 days for all assessed ocular endpoints (fundoscopy, ERG, OCT and histological assessments). In addition, toxicity of (S)-3-amino-4-(difluoromethylenyl)cyclopent-1-ene-1-carboxylic acid was assessed in non-GLP single-dose and repeat dose toxicity studies in rats and dogs. In 10-day dosing in rats, (S)-3-amino-4-(difluoromethylenyl)cyclopent-1-ene-1-carboxylic acid doses up to 15 mg/kg in male and 10 mg/kg in female were well tolerated. In 10-day dosing in male and female dogs, (S)-3-amino-4-(difluoromethylenyl)cyclopent-1-ene-1-carboxylic acid was well tolerated with doses up to 1.0 mg/kg. (S)-3-amino-4-(difluoromethylenyl)cyclopent-1-ene-1-carboxylic acid was not genotoxic in the Ames assay. The Ames assay is a well-known method that uses several strains of the bacterium Salmonella typhimurium to test whether a given chemical can cause mutations in the DNA of the test organism. No CNS or cardiovascular safety liabilities were identified in an Irwin study in mice. The Irwin observation test is commonly used to evaluate the effects of a new substance on behavior and physiological function. Roux et al., Curr Protoc Pharmacol. 2005 Jan. 1; Chapter 10:Unit 10.

No evidence of genetic toxicity or hERG channel activity resulting from (S)-3-amino-4-(difluoromethylenyl)cyclopent-1-ene-1-carboxylic acid was identified in vitro. In adult mice, an acute no-observed adverse-effect level (NOAEL) of 6 mg/kg by mouth was determined by evaluating behavioral neurotoxicity in naive animals. Administration of (S)-3-amino-4-(difluoromethylenyl)cyclopent-1-ene-1-carboxylic acid at 1 mg/kg/d by mouth for 45 days in a separate cohort showed the compound to be well tolerated, with no reduction in body weight gain relative to control, no gross behavioral deficits, and no impact on retinal function as measured by electroretinogram (i.e., retinal B-wave amplitudes). These findings stand in contrast to those from a head-to-head comparison with vigabatrin, where a dose with considerably less GABA-AT inhibitory activity resulted in a marked reduction in weight and reductions in retinal B-wave amplitudes. Based on these findings, it is clear that the inhibition of GABA-AT by (S)-3-amino-4-(difluoromethylenyl)cyclopent-1-ene-1-carboxylic acid is not necessarily associated with vigabatrin-like retinal toxicity.

Conversion of the above-doses to human doses according to Reagan-Shaw et al., FASEB J. 2008 March; 22(3):659-61 yields the following:

15 mg/kg rat=2.4 mg/kg human

10 mg/kg rat=1.62 mg/kg human

1 mg/kg/day rat=0.48 mg/kg/day hE man

1 mg/kg dog=1.54 mg/kg human

6 mg/kg mouse=0.49 mg/kg human

1 mg/kg/day mouse=0.08 mg/kg/day human

### Example 2

Pharmacokinetic and Metabolic Profile of (S)-3-amino-4-(difluoromethylenyl)cyclopent-1-ene-1-carboxylic acid

Following oral administration of (S)-3-amino-4-(difluoromethylenyl)cyclopent-1-ene-1-carboxylic acid in both rat and dog, the time of maximum concentration (Tmax) was 15-30 minutes, and the elimination half-life (t½) was approximately 75-90 minutes. Dogs had higher exposure (AUC basis) than rats for equivalent oral doses. In vitro studies in hepatocytes show clearance of (S)-3-amino-4-(difluoromethylenyl)cyclopent-1-ene-1-carboxylic acid was significantly greater in rat than dog; intrinsic clearance (CIᵢₙₜ)=24.3 (rat), 13.1 (human), and 8.97 mL/min/kg (dog). (S)-3-amino-4-(difluoromethylenyl)cyclopent-1-ene-1-carboxylic acid was minimally bound (4 to 20%) to proteins in rat, dog, and human plasma at 37° C.

### Example 3

### Prospective Assessment of the Efficacy of (S)-3-Amino-4-(difluoromethylenyl)cyclopent-1-ene-1-carboxylic acid in Mouse Models of Medulloblastoma and Leptomeningeal Metastasis

Xenograft models are to be established according to Martirosian et al., 2021, Cell Reports 35, 109302. Accordingly, animal experiments will be performed on 9-10 week old female athymic nude mice, housed in ventilated cages (3-5 animals per cage) (n=20). Animals will be housed in an animal room designated for immuno-deficient mice, along with all cages, bedding, and feed being sterilized to ensure a pathogen-free environment. Animals will be monitored and determined to be healthy and pain-free before and after experiments are performed. Animals will not be used in other procedures before transplantation surgeries. All animals will be humanely euthanized either when reaching a specific time point or upon presentation of signs of morbidity, including development of tumor symptoms (paralysis, hydrocephalus, weight loss, tilted head).

Three medulloblastoma xenograft models (cohorts) will be established using D283 cells which are a rapidly dividing human medulloblastoma cell line (commercially available from the American Type Culture Collection (ATCC)) and Med-2112FH (MD cells commercially available from Brain Tumor Resource Laboratory of Fred Hutchinson Cancer Research): (1) primary tumor models with 2×105 D283 Scrambled, GABA-AT Knock-Down (KD), and GABA-AT Over Expression (OE) medulloblastoma cells transplanted into the cerebellum, (2) competitive leptomeningeal models with 1×105 D283 GABA-AT KD and 1×105 GABA-AT OE medulloblastoma cells transplanted into the cisterna magna, and (3) a clinically relevant dual-injection model depicting primary and metastatic medulloblastoma with 1×105 Med-2112FH injected into both the cerebellum and the cisterna magna. Mice will be initially anesthetized under 5% isoflurane (Vetone, Cat #502017) and maintained at 2%-2.5% isoflurane. To model primary disease, tumor cells will be injected into the cerebellum utilizing a stereotaxic frame. Cells will be transplanted at _6.47 Bregma, 1 mm lateral of the sagittal suture, and 1 mm into the cerebellum. To model leptomeningeal disease, surgery set-up mirrors cerebellar injections, however, cells will be injected into the cisterna magna as previously shown (Xavier et al., 2018 J. Vis. Exp. 135, 57378.). All transplanted lines will express luciferase for in vivo bioluminescent imaging (BLI) post-transplantation. BLI will be performed 3 days and 7 days post-injection, and once every week afterward for the single-injection models. Mice will be monitored for presentation of tumor burden related symptoms and humanely euthanized when indicated. For the dual-injection models, BLI will be performed 3 days post-injection, and every 3-4 days afterward. Mice will be euthanized 21 days post-transplantation to observe immediate effects of the leptomeningeal microenvironment on medulloblastoma cells.

Three treatment regimens will be utilized for the study and will include once/daily, twice/daily or thrice/daily IP dosing of (S)-3-amino-4-(difluoromethylenyl)cyclopent-1-ene-1-carboxylic acid in saline at 1, 3, 5, 6, 10, 15, 20, and 40 mg/kg and saline as vehicle control at a volume of 3 ml/kg body weight in a randomized crossover design. Each group of animals will receive chemotherapeutic treatment for a minimum of three cycles and maintained to determine outcomes. Each of the cohorts will be evaluated to compare increase in survival times compared to untreated animals. Qualitative symptoms to be observed in the mouse model include irregular stride length, impaired cranial nerve function and decreased motor coordination and performance. Several quantitative behavioral assays will include a mouse rotarod, a forced air challenge, a screen inversion test, a horizontal wire test, and stride length analysis. Magnetic resonance imaging (MRI) will be used to confirm and monitor tumor growth and as an anatomical biomarker for therapeutic response.

It should be understood that the examples and embodiments provided herein are exemplary examples and embodiments. Those skilled in the art will envision various modifications of the examples and embodiments that are consistent with the scope of the disclosure herein. Such modifications are intended to be encompassed by the claims.

What is claimed is:

1. A method for inhibiting cancer proliferation and metastasis, comprising administering a composition comprising from about 0.0001 mg/kg to about 30 mg/kg (S)-3-amino-4-(difluoromethylenyl)cyclopent-1-ene-1-carboxylic acid or a pharmaceutically acceptable salt thereof to a subject in need thereof.

2. The method for inhibiting cancer proliferation and metastasis according to claim 1, wherein the composition comprises from about 0.1 mg/kg to 15 mg/kg of (S)-3-amino-4-(difluoromethylenyl)cyclopent-1-ene-1-carboxylic acid or a pharmaceutically acceptable salt thereof.

3. The method for inhibiting cancer proliferation and metastasis according to claim 2, wherein the composition comprises from about 0.2 mg/kg to 10 mg/kg of (S)-3-amino-4-(difluoromethylenyl)cyclopent-1-ene-1-carboxylic acid or a pharmaceutically acceptable salt thereof.

4. The method for inhibiting cancer proliferation and metastasis according to claim 3, wherein the composition comprises from about 0.5 mg/kg to 6.5 mg/kg of (S)-3-amino-4-(difluoromethylenyl)cyclopent-1-ene-1-carboxylic acid or a pharmaceutically acceptable salt thereof.

5. The method for inhibiting cancer proliferation and metastasis according to claim 1, wherein the cancer is medulloblastoma, glioma, breast cancer, squamous cell cancer, melanoma, lung cancer, cancer of the peritoneum, hepatocellular cancer, gastric cancer, pancreatic cancer, glioblastoma, cervical cancer, ovarian cancer, testicular cancer, bladder cancer, colorectal cancer, endometrial or uterine carcinoma, salivary gland carcinoma, kidney or renal cancer, prostate cancer, vulval cancer, thyroid cancer, head and neck cancer, B-cell lymphoma, chronic lymphocytic leukemia, acute lymphoblastic leukemia, hairy cell leukemia, leptomeningeal carcinomatosis, or chronic myeloblastic leukemia and subtypes thereof.

6. The method for inhibiting cancer proliferation and metastasis according to claim 1, wherein the subject is human.

7. The method for inhibiting cancer proliferation and metastasis according to claim 1, wherein the subject is also undergoing one or more cancer therapies selected from the group consisting of surgery, chemotherapy, radiotherapy, thermotherapy, immunotherapy, hormone therapy, or laser therapy.

8. The method for inhibiting cancer proliferation and metastasis according to claim 1, wherein the composition is administered enterally or parenterally.

9. The method for inhibiting cancer proliferation and metastasis according to claim 8, wherein the composition is administered orally, rectally, transdermally, intramuscularly, intravenously, subcutaneously, intraperitoneally, or intrathecally.

10. A method for treating cancer, comprising administering a composition comprising from about 0.0001 mg/kg to about 30 mg/kg (S)-3-amino-4-(difluoromethylenyl)cyclopent-1-ene-1-carboxylic acid or a pharmaceutically acceptable salt thereof to a subject in need thereof, thereby treating the cancer.

11. The method treating cancer according to claim 10, wherein the composition comprises from about 0.1 mg/kg to about 15 mg/kg of (S)-3-amino-4-(difluoromethylenyl)cyclopent-1-ene-1-carboxylic acid or a pharmaceutically acceptable salt thereof.

12. The method treating cancer according to claim 11, wherein the composition comprises from about 0.2 mg/kg to about 10 mg/kg of (S)-3-amino-4-(difluoromethylenyl)cyclopent-1-ene-1-carboxylic acid or a pharmaceutically acceptable salt thereof.

13. The method treating cancer according to claim 12, wherein the composition comprises from about 0.5 mg/kg to about 6.5 mg/kg of (S)-3-amino-4-(difluoromethylenyl)cyclopent-1-ene-1-carboxylic acid or a pharmaceutically acceptable salt thereof.

14. The method treating cancer according to claim 10, wherein the cancer is medulloblastoma, glioma, breast cancer, squamous cell cancer, melanoma, lung cancer, cancer of the peritoneum, hepatocellular cancer, gastric cancer, pancreatic cancer, glioblastoma, cervical cancer, ovarian cancer, testicular cancer, bladder cancer, colorectal cancer, endometrial or uterine carcinoma, salivary gland carcinoma, kidney or renal cancer, prostate cancer, vulval cancer, thyroid cancer, head and neck cancer, B-cell lymphoma, chronic lymphocytic leukemia, acute lymphoblastic leukemia, hairy cell leukemia, leptomeningeal carcinomatosis, or chronic myeloblastic leukemia and subtypes thereof.

15. The method treating cancer according to claim 10, wherein the subject is human.

16. The method for treating cancer according to claim 10, wherein the subject is also undergoing one or more cancer therapies selected from the group consisting of surgery, chemotherapy, radiotherapy, thermotherapy, immunotherapy, hormone therapy, or laser therapy.

17. The method for inhibiting cancer proliferation and metastasis according to claim 10, wherein the composition is administered enterally or parenterally.

18. The method for inhibiting cancer proliferation and metastasis according to claim 17, wherein the composition is administered orally, rectally, transdermally, intramuscularly, intravenously, subcutaneously, intraperitoneally, or intrathecally.
